(19) United States
(12) Patent Application Publication
(10) Pub. No.: US 2026/0085032 A1
(43) Pub. Date: Mar. 26, 2026

(54) NOVEL SURFACTANTS DERIVED FROM GLYCIDYL ESTERS OF ROSIN ACIDS

(71) Applicant: LAMBERTI SPA, Albizzate (IT)

(72) Inventors: Cristina PICCO, Oleggio (IT); Mauro MAPELLI, Somma Lombardo (IT); Alessandro D'ALOIA, Novi Ligure (IT); Filippo QUAGLIA, Novate Milanese (IT); Dario FORNARA, Novara (IT); Federico DE PELLEGRINI, Albizzate (IT)

(73) Assignee: LAMBERTI SPA, Albizzate (IT)

(21) Appl. No.: 19/106,845

(22) PCT Filed: Sep. 5, 2023
(86) PCT No.: PCT/EP2023/074309

§ 371 Date: Feb. 26, 2025

(30) Foreign Application Priority Data
Sep. 8, 2022 (IT) 102022000018282

(51) Int. Cl. C07C 69/587; A01N 37/06; A01N 41/02; A01N 57/12; C07C 309/70; C07F 9/09

(52) U.S. Cl. CPC C07C 69/587; A01N 37/06; A01N 41/02; A01N 57/12; C07C 309/70; C07F 9/09

(57) ABSTRACT

A surfactant of Formula (I), wherein: R₁=residue derived from a rosin acid; X=H, C₁-C₂₀-alkyl, P=O(OR₂)(OR₃) or SO₃R₄; Y=H, P=O(OR₂)(OR₃), SO₃R₄; A=(EO)r-(PO)s, or a residue derived from a diol or a polyol selected among polyglycerol, propane-1,3-diol, glycerol, trimethylolethane, trimethylolpropane, sorbitan, sorbitol, isosorbide, erythritol, threitol, pentaerythritol, arabitol, xylitol, ribitol, fucitol, mannitol, galactitol, iditol, inositol, volemitol or glucose; R₂, R₃ and R₄=hydrogen, an alkali metal, ammonium ion, a protonated amine, a quaternary organic ammonium or an alkaline earth metal; EO=CH₂CH₂O; PO=CH₂CH(CH₃)O; n is an integer number ranging from 1 to 10; p and q are integer numbers ranging from 0 to 100; r and s are integer numbers ranging from 0 to 100, with the proviso that the sum r+s is at least 1.

### TECHNICAL FIELD

The present invention relates to novel surfactants derived from glycidyl esters of rosin acids and to agrochemical compositions containing them.

### BACKGROUND OF THE ART

Surfactants containing aromatic groups in their hydrophobic portion are important in many different applications due to their unique functionality, high performance and low cost. For example, phenols may be reacted with olefins (e.g. 1-nonene or 1-octene) to produce alkylphenol hydrophobes, while styrene may be reacted with phenol to produce mono-, di- and tristyrylphenols. These aromatic-based hydrophobes may then be ethoxylated to produce surfactants and employed in a variety of applications such as agriculture, home and personal care, oil and gas industry, inks, paints and coatings.

However, both of these surfactant classes based on aromatic hydrophobes exhibit undesirable properties. In particular, recent studies have suggested that these conventional ethoxylates experience delayed or deficient biodegradation and can generate biodegradation products, such as alkylphenols and tristyrylphenol. Alkylphenols have been found to be endocrine disruptors while alkylphenols and tristyrylphenol are known to be persistent environmental pollutants. In addition, styrene used in the production of mono-, di- and tristyrylphenol is known to also exhibit undesirable toxicity properties while being highly flammable. Thus, the storage and handling of this compound in an industrial setting poses significant safety risks and is becoming highly disfavored.

Considering the hazardous and potential ecological effects due to manufacturing, processing and distribution of ethoxylates derived from the above described aromatic-based hydrophobes, there remains a significant need to identify, develop and employ alternatives to these conventional hydrophobes and their alkoxylated derivatives.

In addition, alkylphenols and tristyrylphenol are derived from petrochemical resources. Therefore, there is also a need to find alternatives to alkylphenol- or tristyrylphenol-based surfactants that are partially or fully derived from renewable resources.

In this context, rosin acids, particularly their glycidyl esters, represent a suitable starting point to develop surfactants that can replace the classical alkylphenol- or tristyrylphenol-based surfactants.

The preparation of glycidyl esters of fatty or rosin acids is well known in the art, as they can be obtained by reaction of fatty or rosin acids with an epihalohydrin.

WO2021073765 relates to α,α-branched alkane carboxylic acids glycidyl esters which derived from rosin and or hydrogenated rosin reacted with an epihalohydrin.

WO2021188613 concerns methods for preparing tall oil derived glycidyl esters.

In the prior art rosin acids have been used as raw materials to prepare surfactants.

U.S. Pat. No. 1,984,713 describes a process for preparing abietyl esters of a sulpho-propane-diol.

U.S. Pat. No. 1,984,714 relates to a process for preparing sulpho-alkyl esters of abietic acids.

U.S. Pat. No. 2,950,272 describes the preparation of polyoxyalkylene esters of tall oil rosin acids.

U.S. Pat. No. 3,259,615 relates to the preparation of 3-amino-2-hydroxypropyl esters of rosin acids and quaternary ammonium salts thereof, and their use as antifungal, antibacterial, herbicidal, fungicidal and nematocidal agents.

U.S. Pat. No. 3,541,134 relates to reaction products of rosin-fumaric acid adducts and alkylene oxides.

U.S. Pat. No. 4,312,631 describes surface-active compounds obtained by oxyalkylating natural rosin acids, reacting the alkylene oxide addition products thus obtained with maleic acid anhydride, and then adding alkali metal sulfite or alkaline earth metal sulfites or hydrogen sulfites to the maleic acid semiesters.

U.S. Pat. No. 4,238,380 describes the preparation of disodium ethoxylated rosin half esters of sulfosuccinic acid and their use as foam stabilizers.

U.S. Pat. No. 4,260,550 relates to modified rosin esters obtained by reacting abietic acid with maleic anhydride, and then reacting the maleated rosin intermediate with a nonionic surfactant.

EP0159794 describes novel surfactants that are sulfosuccinate half ester of an alkoxylated, fortified rosin acids.

U.S. Pat. No. 5,552,519 describes surfactants obtainable by esterifying a rosin with an alcohol, grafting the rosin ester with an unsaturated carboxylic acid or anhydride, and esterifying the grafted rosin ester with a polyethylene glycol.

CN1180075 describes a method for preparing polyglycol ester abietate non-ionic surfactant.

However, in the prior art there is no reference to surfactants prepared starting from glycidyl esters of rosin acids.

### SUMMARY OF THE INVENTION

It is therefore an object of the present invention a surfactant of Formula (I):

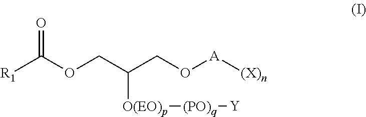

wherein:
R₁=residue derived from a rosin acid

$$X = H , {C}_{1} - {C}_{20} - alkyl , P = O ( {OR}_{2} ) ( {OR}_{3} ) or {SO}_{3} {R}_{4}$$

$$Y = H , P = O ( {OR}_{2} ) ( {OR}_{3} ) , {SO}_{3} {R}_{4}$$

A=(EO)r-(PO)s, or a residue derived from a diol or a polyol selected among polyglycerol, propane-1,3-diol, glycerol, trimethylolethane, trimethylolpropane, sorbitan, sorbitol, isosorbide, erythritol, threitol, pentaerythritol, arabitol, xylitol, ribitol, fucitol, mannitol, galactitol, iditol, inositol, volemitol or glucose
R₂, R₃ and R₄=hydrogen, an alkali metal, ammonium ion, a protonated amine, a quaternary organic ammonium or an alkaline earth metal

$$EO = {CH}_{2} {CH}_{2} O$$

$$PO = {CH}_{2} CH ( {CH}_{3} ) O$$

n is an integer number ranging from 1 to 10, preferably from 1 to 5
p and q are integer numbers ranging from 0 to 100, preferably from 2 to 40, more preferably from 4 to 20
r and s are integer numbers ranging from 0 to 100, preferably from 2 to 40, more preferably from 4 to 20, with the proviso that the sum r+s is at least 1.

A further object of the present invention is an agrochemical composition comprising:
a) at least one agrochemically active ingredient;
b) at least one surfactant of Formula (I).

### DETAILED DESCRIPTION OF THE INVENTION

According to the present invention, in Formula (I) R₁ is a residue derived from a rosin acid. Rosin acids are C₂₀ mono-carboxylic acids with a core having a fused carbocyclic ring system comprising double bonds that vary in number and location. Particularly, rosin acids are derived from rosin, which is a blend of at least eight monocarboxylic acids (abietic acid, palustric acid, dehydroabietic acid, neo-abietic acid, levo-pimaric acid, pimaric acid, sandaracopimaric acid and isopimaric acid). Abietic acid is usually the primary species present in rosin and the other seven acids are isomers thereof.

As used herein, the term “rosin acid” include also chemically modified rosin acids such as partially or fully hydrogenated rosin acids, partially or fully dimerized rosin acids, disproportionated rosin acids, or combinations thereof. The term “rosin acid” includes also rosin acid derivatives obtained by Diels-Alder reaction of a rosin acid with a dienophile such as maleic anhydride, fumaric acid, acrylonitrile, itaconic anhydride, or acrylic acid.

The rosin acids of the invention are obtained from crude tall oil, rosin, tall oil rosin, gum tree rosin, wood rosin, softwood rosin, hardwood rosin, distilled tall oil, derivatives thereof, or a combination thereof.

According to the invention, suitable blends of rosin acids have a content of fatty acids less than 15% by weight, preferably less than 4% by weight, more preferably less than 2% by weight.

According to a preferred embodiment, in Formula (I) R₁ is a residue derived from a rosin acid selected among abietic acid, palustric acid, dehydroabietic acid, neo-abietic acid, levo-pimaric acid, pimaric acid, sandaracopimaric acid, isopimaric acid or mixtures thereof.

According to the invention, in Formula (I) A is (EO)r-(PO)s, or a residue derived from the removal of two hydroxyl hydrogens of a diol or two or more hydroxyl hydrogens of a polyol. Said diol or polyol is selected among polyglycerol, propane-1,3-diol, glycerol, trimethylolethane, trimethylolpropane, sorbitan, sorbitol, isosorbide, erythritol, threitol, pentaerythritol, arabitol, xylitol, ribitol, fucitol, mannitol, galactitol, iditol, inositol, volemitol or glucose.

Suitable polyglycerols have a mean degree of condensation of 1-20, preferably of 2-10 and more preferably of 2.5-8 is used. The mean degree of condensation N can be determined here on the basis of the OH number (OHN, in mg KOH/g) of the polyglycerol and is linked thereto according to:

$$N = \frac{112200 - 18 \cdot OHN}{75 \cdot OHN - 56100}$$

The OH number of the polyglycerol can be determined according to the methods DGF C-V 17 a (53), Ph. Eur. 2.5.3 Method A, or DIN 53240.

Preferred polyglycerols according to the invention are especially those which have an OH number of 1829 to 824, more preferably of 1352-888 and especially preferably of 1244-920 mg KOH/g.

According to a preferred embodiment, A is (EO)r-(PO)s, wherein EO=CH₂CH₂O and PO=CH₂CH(CH₃)O, r and s are integer numbers ranging from 0 to 100, preferably from 2 to 40, more preferably from 4 to 20, with the proviso that the sum r+s is at least 1.

According to the invention, R₂, R₃ and R₄ are hydrogen, an alkali metal, ammonium ion, a protonated amine, a quaternary organic ammonium or an alkaline earth metal. Examples of suitable amines are alkyl amines and alkanol amines.

The surfactants of the present invention are prepared starting from glycidyl esters of rosin acids or the diols obtained by hydrolysis of the epoxide group present in said glycidyl esters. The preparation of glycidyl esters of rosin acids is well known in the art: they are typically prepared by reaction of a rosin acid with epichlorohydrin, as described for example in JPS5560575, JPH0515751, JPH09143430, WO2021073764 or WO2021188613.

When starting from a glycidyl ester of a rosin acid, the process for preparing surfactants of the present invention comprises a first step, wherein the epoxide of the glycidyl ester is opened by reaction with a polyalkylene glycol (such as polyethylene glycol or polypropylene glycol), a polyalkylene glycol monoalkyl ether (such as methoxy polyethylene glycol or ethoxy polyethylene glycol), a diol or a polylol selected among polyglycerol, propane-1,3-diol, glycerol, trimethylolethane, trimethylolpropane, sorbitan, sorbitol, isosorbide, erythritol, threitol, pentaerythritol, arabitol, xylitol, ribitol, fucitol, mannitol, galactitol, iditol, inositol, volemitol or glucose. Suitable polyalkylene glycols and polyalkylene glycol monoalkyl ethers have a molecular weight comprised between 100 and 10,000 g/mol, preferably 300 and 5,000 g/mol. The reaction is conducted at temperature comprised between 10° and 160° C., preferably from 120 to 140° C., in the presence of a catalyst (such as for example an alkali metal alkoxide).

The epoxide of the glycidyl ester is opened by reaction preferably with a polyalkylene glycol or a polyalkylene glycol monoalkyl ether, more preferably with a polyalkylene glycol monoalkyl ether.

The opening of the epoxide leads to the formation of a hydroxyl group, which can undergo subsequent reactions (such as alkoxylation and/or esterification to form phosphate or sulfate esters.)

Alkoxylation (reaction with, e.g., ethylene oxide and/or propylene oxide) is carried out at a temperature comprised between 10° and 160° C. and in the presence of a conventional catalyst, for example an alkali metal hydroxide.

Phosphate esters are obtained by reaction with a phosphating agent, e.g. P₂O₅ or phosphoric acid or a derivative thereof, for example a polyphosphoric acid, such as tetraphosphoric acid. The esterification into phosphate esters is advantageously carried out at a temperature from 35 to 110° C.

Sulfate esters are obtained by reaction with a sulfating agents. A particularly preferred sulfating agent is sulfamic acid, which leads to the formation a sulfate ester neutralized with an ammonium ion. The esterification into sulfate esters is advantageously carried out at a temperature from 70 to 140° C.

Alternatively, when using the diol of the glycidyl ester of a rosin acid as raw material, the process for preparing the surfactants of the present invention comprises a first step, wherein said diol undergoes alkoxylation (with ethylene oxide and/or propylene oxide). Subsequently, the alkoxylated diol can be further reacted to obtain phosphate esters or sulfate esters. The surfactants of the present invention are suitable for use in various application fields, such as agriculture, home and personal care, oil and gas industry, inks, paints and coatings. Particularly, the surfactants of the present invention were found to be useful as dispersing agents and emulsifiers for agrochemical compositions.

When used as dispersing agents, the surfactants of the present invention are particularly suitable for preparing aqueous suspension concentrate (SC) formulations.

When used as emulsifiers, the surfactants of the present invention are particularly suitable for preparing emulsifiable concentrate (EC) formulations.

The agrochemical composition of the present invention comprises:
a) from 0.1 to 99 wt % (% by weight), preferably from 1 to 70 wt %, more preferably from 5 to 60 wt % of at least one agrochemically active ingredient;
b) from 0.01 to 40 wt % (% by weight), preferably from 0.1 to 15 wt %, more preferably from 0.5 to 10 wt % of at least one surfactant of Formula (I).

According to a preferred embodiment, the agrochemical composition of the present invention is a suspension concentrate (SC) or an emulsifiable concentrate (EC).

Suitable agrochemically active ingredients of the agrochemical composition are substantially solid materials that are water- or oil-insoluble at room temperature such as, for example, fungicides, bactericides, insecticides, acaricides, nematicides, herbicides, aracnicides, insect growth regulators, repellents, plant growth regulators, plant nutrients or mixtures thereof. Preferably, the agrochemical active ingredient is selected among fungicides, bactericides, insecticides, acaricides, nematicides, herbicides, aracnicides or mixtures thereof.

Examples of fungicides which may be mentioned are:

2-anilino-4-methyl-6-cyclopropyl-pyrimidine; 2′,6′-dibromo-2-methyl-4′-trifluoro-methoxy-4′-trifluoromethyl-1,3-thiazole-5-carboxanilide; 2,6-dichloro-N-(4-trifluoro-methylbenzyl)-benzamide; (E)-2-methoximino-N-methyl-2-(2-phenoxyphenyl)-acetamide; 8-hydroxyquinoline sulphate; methyl(E)-2-{2-[6-(2-cyanophenoxy)-pyrimidin-4-yloxy]-phenyl}-3-methoxyacrylate; methyl(E)methoximino[alpha-(o-tolyloxy)-o-tolyl]-acetate; 2-phenylphenol (OPP), aldimorph, ampropylfos, anilazine, azaconazole, benalaxyl, benodanil, benomyl, binapacryl, biphenyl, bitertanol, blasticidin-S, bromuconazole, bupirimate, buthiobate, calcium polysulphide, captafol, captan, carbendazim, carboxin, quinomethionate, chloroneb, chloropicrin, chlorothalonil, chlozolinate, cufraneb, cymoxanil, cyproconazole, cyprofuram, carpropamide, dichlorophen, diclobutrazole, dichlofluanid, diclomezin, dicloran, diethofencarb, difenoconazole, dimethirimol, dimethomorph, diniconazole, dinocap, diphenylamine, dipyrithion, ditalimfos, dithianon, dodine, drazoxolon, edifenphos, epoxyconazole, ethirimol, etridiazole, fenarimol, fenbuconazole, fenfuram, fenitropan, fenpiclonil, fentin acetate, fentin hydroxide, ferbam, ferimzone, fluazinam, fludioxonil, fluoromide, fluquinconazole, flusilazole, flusulfamide, flutolanil, flutriafol, folpet, fosetyl-aluminium, fthalide, fuberidazole, furalaxyl, fenhexamide, guazatine, hexachlorobenzene, hexaconazole, hymexazole, imazalil, imibenconazole, iminoctadine, iprobenfos (IBP), iprodion, isoprothiolan, iprovalicarb, kasugamycin, copper preparations, such as: copper hydroxide, copper naphthenate, copper oxychloride, copper sulphate, copper oxide, oxine-copper and Bordeaux mixture; mancopper, mancozeb, maneb, mepanipyrim, mepronil, metalaxyl, metconazole, methasulfocarb, methfuroxam, metiram, metsulfovax, mydobutanil, nickel dimethyldithiocarbamate, nitrothalisopropyl, nuarimol, ofurace, oxadixyl, oxamocarb, oxycarboxine, pefurazoate, penconazole, pencycuron, phosdiphen, pimaricin, piperalin, polyoxine, probenazole, prochloraz, procymidon, propamocarb, propiconazole, propineb, pyrazophos, pyrifenox, pyrimethanil, pyroquilon, quintozene (PCNB), quinoxyfen, sulphur and sulphur preparations, tebuconazole, tecloftalam, tecnazene, tetraconazole, thiabendazole, thicyofen, thiophanate-methyl, thiram, toldlophos-methyl, tolylfluanid, triadimefon, triadimenol, triazoxide, trichlamide, tricyclazole, tridemorph, trifiumizole, triforin, triticonazole, trifioxystrobin, validamycin A, vinclozolin, zineb, ziram, ciproconazole, dodine, fenamidone, fenexamide, fluopicolide, fluoxastrobin, fosetyl-aluminium, iprovalicarb, pencycuron, prothioconazole, spiroxamina, triadimenol, trifloxystrobin, azoxystrobin, acibenzolar-S-methyl, ciprodinil, mandipropamid, fenpropidin, boscalid, kresoxim-methyl, pyraclostrobin, dimetomorf, fenpropimorf, metraphenone, tolclofos-methyl and 2-[2-(1-chloro-cyclopropyl)-3-(2-chlorophenyl)-2-hydroxypropyl]-2,4-dihydro-[1,2,4]-triazole-3-thione.

Examples of bactericides which may be mentioned are:

bronopol, dichlorophen, nitrapyrin, nickel dimethyldithiocarbamate, kasugamycin, octhilinon, furancarboxylic acid, oxytetracycline, probenazole, streptomycin, tecloftalam, copper sulphate and other copper preparations.

Examples of insecticides, acaricides and nematicides which may be mentioned are:

abamectin, acephate, acrinathrin, alanycarb, aldicarb, alphamethrin, amitraz, avermectin, AZ 60541, azadirachtin, azinphos A, azinphos M, azocyclotin, Bacillus thuringiensis, 4-bromo-2-(4-chlorophenyl)-1-(ethoxymethyl)-5-(trifluoromethyl)-pyrrole-3-carbonitrile, bendiocarb, benfuracarb, bensultap, betacyfluthrin, bifenthrin, BPMC, brofenprox, bromophosa, bufencarb, buprofezin, butocarboxine, butylpyridaben, cadusafos, carbaryl, carbofuran, carbophenothion, carbosulfan, cartap, chloethocarb, chlaretoxyfos, chlorfenvinphos, chlorfluazuron, chlormephos, N-[(6-chloro-3-pyridinyl)-methyl]-N′-cyano-N-methyl-ethaneimidamide, chlorpyrifos, chlorpyrifos M, cis-resmethrin, clocythrin, clofentezin, cyanophos, cycloprothrin, cyfluthrin, cyhalothrin, cyhexatin, cypermethrin, cyromazin, deltamethrin, demeton-M, demeton-S, demeton-S-methyl, diafenthiuron, diazinon, dichlofenthion, dichlorvos, dicliphos, dicrotophos, diethion, diflubenzuron, dimethoate, dimethylvinphos, dioxathion, disulfoton, emamectin, esfen valerate, ethiofencarb, ethion, ethofenprox, ethoprophos, etrimphos, fenamiphos, fenazaquin, fenbutatin oxide, fenitrothion, fenobucarb, fenothiocarb, fenoxycarb, fenpropathrin, fenpyrad, fenpyroximate, fenthion, fenvalerate, fipronil, fluazuron, flucycloxuron, flucythrinate, flufenoxuron, flufenprox, fluvalinate, fonophos, formothion, fosthiazate, fubfenprox, furathiocarb, HCH, heptenophos, bexaflumuron, hexythiazox, imidacloprid, iprobenfos, isazophos, isofenphos, isoprocarb, isoxathion, ivermectin, lambdacybalothrin, lufenuron, malathion, mecarbam, mevinphos, mesulfenphos, metaldehyde, methacrifos, methamidophos, methidathion, methiocarb, methomyl, metolcarb, milbemectin, monocrotophos, moxidectin, naled, NC 184, nitenpyram, omethoate, oxamyl, oxydemethon M, oxydeprofos, parathion AL, parathion ML, permethrin, phenthoate, phorate, phosalon, phosmet, phosphamidon, phoxim, pirimicarb, pirimiphos M, pirimiphos A, profenophos, promecarb, propapbos, propoxur, prothiophos, prothoate, pymetrozine, pyrachlophos, pyridaphenthion, pyresmethrin, pyrethrum, pyridaben, pyrimidifen, pyriproxifen, quinalphos, salithion, ebufos, silafluofen, sulfotep, sulprofos, tebufenozide, tebufenpyrad, tebupirimiphos, teflubenzuron, tefluthrin, temephos, terbam, terbufos, tetrachlorvinphos, thiacloprid, thiafenox, thiamethoxam, thiodicarb, thiofanox, thiomethon, thionazine, thuringiensin, tralomethrin, transfluthrin, triarathen, triazophos, triawron, trichlorfon, triflumuron, trimethacarb, vamidothion, XMC, xylylcarb, zetamethrin, ethoprophos, fenpyroximate, methoxyfenozide, spinosad, spirodiclofen, thiacloprid, cypermethrine, alphacypermethrine, alphametrine and metaflumizone.

Examples of herbicides which may be mentioned are:

anilides, such as, for example, diflufenican and propanil; arylcarboxylic acids, such as, for example, dichlorpicolinic acid, dicamba and picloram; aryloxyalkanoic acids, such as, for example, 2,4-D, 2,4-DB, 2,4-DP, Fluroxypyr, MCPA, MCPP and triclopyr; aryloxy-phenoxy-alkanoic acid esters, such as, for example, diclofop-methyl, fenoxapropethyl, Fluazifopbutyl, haloxyfop-methyl and quizalofop-ethyl; azinones, such as, for example, chloridazon and norflurazon; carbamates, such as, for example, chlorpropham, desmedipham, phenmedipham and propham; chloroacetanilides, such as, for example, alachlor, acetochlor, butachlor, metazachlor, metolachlor, pretilachlor and propachlor; dinitroarilines, such as, for example, oryzalin, pendimethalin and trifluralin; diphenyl ethers, such as, for example, acifluorfen, bifenox, fluoroglycofen, fomesafen, halosafen, lactofen and oxyfluorfen; ureas, such as, for example, chlortoluron, diuron, fluometuron, isoproturon, linuron and methabenzthiazuron; hydroxylamines, such as, for example, alloxydim, clethodim, cycloxydim, sethoxydim and tralkoxydim; imidazolinones, such as, for example, imazethapyr, imazamethabenz, imazapyr and imazaquin; nitriles, such as, for example, bromoxynil, dichlobenil and ioxynil; oxyacetamides, such as, for example, mefenacet; sulphonylureas, such as, for example, amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron-methyl-sodium, foramsulfuron, halosulfuron-methyl, imazosulfuron, iodosulfuron-methyl-sodium, metsulfuron-methyl, nicosulfuron, oxasulfuron, primisulfuron-methyl, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl; triketones such as, for example, mesotrione, tembotrione, topramezone, fenquinotrione and sulcotrione; thiocarbamates, such as, for example, butylate, cycloate, diallate, EPTCL, esprocarb, molinate, prosulfocarb, thiobencarb and triallate; triazines, such as, for example, atrazine, cyanazine, simazine, simetryne, terbutryne and terbutylazine; triazinones, such as, for example, hexazinon, metarnitron and metribuzin; others, such as, for example, aminotriazole, benfuresate, bentazone, cinmethylin, clomazone, clopyralid, difenzoqual, dithiopyr, ethofumesate, fluorochloridone, glufosinate, glyphosate, isoxaben, pyridate, quinchlorac, quinmerac, sulfosate and tridiphane, aclodifen, bap, bispyribac-sodium, ethoxysulfuron, flufenacet, isoxadifen ethyl, isoxaflutole, mefenpyr diethyl, florasulam, clodinafop propargyl, pinoxaden, trinexapac ethyl, dimethenamide-P, imazamox, profoxydim, tepraloxidim. In addition, 4-amino-N-(1,1-dimethylethyl)-4,5-dihydro-3-(1-methylethyl)-5-oxo-1H-1,2,4-triazole-carboxamide and 2-((((4,5-dihydro-4-methyl-5-oxo-3-propoxy-1H-1,2,4-triazol-1-yl)carbonyl)amino)sulfonyl) methyl sodium benzoate may be mentioned.

Examples of plant growth regulators which may be mentioned are chlorocholine chloride and ethephon.

Examples of repellents which may be mentioned are diethyl-toluamide, ethylhexane-diol and buto-pyronoxyl.

Examples of plant nutrients which may be mentioned are customary inorganic or organic fertilizers for supplying plants with macro- and/or micro-nutrients, such as: ammonium salts, such as ammonium sulfate, ammonium bisulfate, ammonium salts of carboxylic acids, ammonium chloride, ammonium carbonate, ammonium phosphate, urea and urea derivatives; phosphate sources, such as phosphoric salts (MAP monoammoniumphosphate, DAP diammoniumphosphate); potash sources, like potassium phosphate and mono- or di-potassium carbonate; compounds containing micronutrients and secondary nutrients like Zinc, Manganese, Magnesium, Iron, Calcium, Nickel, Molibdenum, Sulfur, Boron, and their chelated salts; polycarboxylic acids, such as citric acid; and mixture thereof; protein derivatives and hydrolyzed proteins and mixture thereof. Preferred plant nutrients are MAP, ammonium sulfate, sulfur and urea.

Other classes of agrochemically active ingredients that are suitable for being formulated as agrochemical compositions will be clearly understood by those skilled in the art or can be found, for example, in “The Pesticide Manual”, 15th edition, The British Crop Protection Council, 2009, and the literature cited therein.

The agrochemical composition may optionally contain one or more additional ingredients known in the art, such as water conditioners, chelating agents, antioxidants, antifoam agents, fillers, wetting agents, dispersing agents, anti-settling agents, spreading agents, pH adjusting agents, binding agents, stabilizers, water, organic solvents, antifreeze agents, penetrants, bioactivators or compatibilizing agents.

The invention is further illustrated by the following examples.

### EXAMPLES

In the following examples, the acid number is measured according standard method ASTM D1980, the percentage of SO₃ is measured according to standard method ASTM D1681, the epoxy equivalent is measured according to standard method ASTM D1652.

### Example 1

### Reaction Product of Glicydyl Ester of Rosin Acids and PEG-800

In a reaction vessel equipped with heating, stirrer, thermometer, a system of introduction of the reagents, such reaction vessel connected to a cooler provided of collector of water, PEG-800, a polyethylene glycol having a molecular weight of 800 g/mol (566 g), is added. The reactor is heated up to 95-105° C. under stirring and vacuum is applied for 1 hour to remove the residual moisture. The vessel is then heated up to 125-130° C. under nitrogen flow and potassium tert-butylate powder (1.94 g) is added. The reaction mixture is maintained at this temperature for about 30 minutes. A first aliquot of glicydyl ester of rosin acids (159 g) is added and the reaction mixture is maintained at this condition for about 90 minutes. A second aliquot of glicydyl ester of rosin acids (159 g) is added. The reaction mixture is maintained at 125-135° C. until the epoxy equivalent is >25000 g/eqEP.

### Example 2

### Phosphate Ester (Triethanolamine Salt) of the Product Prepared in Example 1

In a reaction vessel equipped with heating, stirrer, thermometer, a system of introduction of the reagents, such reaction vessel connected to a cooler provided of collector of water, the reaction product of Example 1 (284.8 g) is added. The reactor is heated to 60° C., under stirring, and hypophosphorous acid 50% in aqueous solution (0.15 g) is added. The vessel is heated up to 95-105° C. and vacuum is applied for 1 hour to remove the residual moisture. The reaction mixture is then cooled down to 35-40° C. under nitrogen flow and phosphoric anhydride (13.7 g) is added in about 4 hours. Once phosphoric anhydride has been loaded completely the reaction mixture is maintained at 38-42° C. for about 30 minutes. Distilled water (1.91 g) is then added and the vessel is heated up to 55-60° C. The reaction mixture is maintained at 55-60° C. until the acid number reaches a value of approximately 55 mg KOH/g. The vessel is then cooled down to 45-50° C. and triethanolamine (35 g) is added keeping under stirrer for 30 minutes.

### Example 3

### Sulfate Ester (Ammonium Salt) of the Product Prepared in Example 1

In a reaction vessel equipped with heating, stirrer, thermometer, a system of introduction of the reagents, such reaction vessel connected to a cooler provided of collector of water, the reaction product of Example 1 (320.91 g) is added. The reactor is heated to 60° C., under stirring, and hypophosphorous acid 50% in aqueous solution (0.17 g) is added. The vessel is heated up to 95-105° C. and vacuum is applied for 1 hour to remove the residual moisture. After bringing back to atmospheric pressure, under nitrogen flow, urea (0.59 g) is added and the vessel is heated up to 115-120° C. At this temperature, sulfamic acid (29.01 g) is added in about 2 hours. The reaction mixture is maintained at 115-120° C. until the percentage of SO₃ reaches a value of approximately 3%.

### Example 4

### Reaction Product of Glicydyl Ester of Rosin Acids and MeOPEG-750

In a reaction vessel equipped with heating, stirrer, thermometer, a system of introduction of the reagents, such reaction vessel connected to a cooler provided of collector of water, MeOPEG-750, a methoxy polyethylene glycol having a molecular weight of 750 g/mol (625.3 g), is added. The reactor is heated up to 95-105° C. under stirring and vacuum is applied for 1 hour to remove the residual moisture. The vessel is then heated up to 125-130° C. under nitrogen flow and potassium tert-butylate powder (2.2 g) is added. The reaction mixture is maintained at this temperature for about 30 minutes. A first aliquot of glicydyl ester of rosin acids (187.35 g) is added and the reaction mixture is maintained at this condition for about 90 minutes. A second aliquot of glicydyl ester of rosin acids (187.35 g) is added. The reaction mixture is maintained at 125-135° C. until the epoxy equivalent is >25000 g/eqEP.

### Example 5

### Phosphate Ester (Triethanolamine Salt) of the Product Prepared in Example 4

In a reaction vessel equipped with heating, stirrer, thermometer, a system of introduction of the reagents, such reaction vessel connected to a cooler provided of collector of water, the reaction product of Example 4 (245 g) is added. The reactor is heated to 60° C., under stirring, and hypophosphorous acid 50% in aqueous solution (0.13 g) is added. The vessel is heated up to 95-105° C. and vacuum is applied for 1 hour to remove the residual moisture. The reaction mixture is then cooled down to 35-40° C. under nitrogen flow and phosphoric anhydride (14.73 g) is added in about 4 hours. Once phosphoric anhydride has been loaded completely the reaction mixture is maintained at 38-42° C. for about 30 minutes. Distilled water (1.14 g) is then added and the vessel is heated up to 55-60° C. The reaction mixture is maintained at 55-60° C. until the acid number reaches a value of approximately 65 mg KOH/g. The vessel is then cooled down to 45-50° C. and triethanolamine (40 g) is added keeping under stirrer for 30 minutes.

### Example 6

### Sulfate Ester (Ammonium Salt) of the Product Prepared in Example 4

In a reaction vessel equipped with heating, stirrer, thermometer, a system of introduction of the reagents, such reaction vessel connected to a cooler provided of collector of water, the reaction product of Example 4 (230 g) is added. The reactor is heated to 60° C., under stirring, and hypophosphorous acid 50% in aqueous solution (0.13 g) is added. The vessel is heated up to 95-105° C. and vacuum is applied for 1 hour to remove the residual moisture. After bringing back to atmospheric pressure, under nitrogen flow, urea (0.44 g) is added and the vessel is heated up to 115-120° C. At this temperature, sulfamic acid (27 g) is added in about 2 hours. The reaction mixture is maintained at 115-120° C. until the percentage of SO₃ reaches a value of approximately 4.5%.

### Example 7

Ethoxylation (with 15 Moles of Ethylene Oxide) of the Diol Derived from Epoxide Opening of the Glycidyl Ester of Rosin Acids

In a stirred stainless steel reactor equipped for temperature measurement and control, pressure measurement, vacuum and inert gas purging, sampling and for introduction of ethylene oxide as a liquid, melted diol of the glicydyl ester of rosin acids (962 g) and potassium hydroxide (35% in aqueous solution, 21.9 g) are added. The reactor content is heated to 120-125° C. and maintained under vacuum and nitrogen flow for 45 minutes to reduce its moisture content to less than 0.1%. The reactor is pressurized with nitrogen to 110-140 kPa and heated to 135° C. Ethylene oxide (1410 g) is then added while the temperature is maintained at 135-145° C. Following a 30 minute period of digestion at reaction temperature, the reaction mixture is cooled to 60° C.

### Example 8

### Phosphate Ester of the Product Prepared in Example 7

In a reaction vessel equipped with heating, stirrer, thermometer, a system of introduction of the reagents, such reaction vessel connected to a cooler provided of collector of water, the reaction product of Example 7 (661.7 g) is added. The reactor is heated to 60° C., under stirring, and hypophosphorous acid (50% in aqueous solution, 0.35 g) is added. The vessel is heated up to 95-105° C. and vacuum is applied for 1 hour to remove the residual moisture. The reaction mixture is then cooled down to 35-40° C. under nitrogen flow and phosphoric anhydride (34.8 g) is added in about 4 hours. Once phosphoric anhydride has been loaded completely the reaction mixture is maintained at 38-42° C. for about 30 minutes. Distilled water (4.46 g) is then added and the vessel is heated up to 55-60° C. The reaction mixture is maintained at 55-60° C. until the acid number reaches a value of approximately 58 mg KOH/g.

### Example 9

### Phosphate Ester (Triethanolamine Salt) of the Product Prepared in Example 8

In a reaction vessel equipped with heating, stirrer, thermometer, a system of introduction of the reagents, such reaction vessel connected to a cooler, the reaction product of Example 8 (320 g) is added. The reactor is heated to 40-50° C., under stirring, and triethanolamine (45 g) is added keeping under stirrer for 30 minutes.

### Example 10

### Sulfate Ester (Ammonium Salt) of the Product Prepared in Example 7

In a reaction vessel equipped with heating, stirrer, thermometer, a system of introduction of the reagents, such reaction vessel connected to a cooler provided of collector of water, the reaction product of Example 7 (317.4 g) is added. The reactor is heated to 60° C., under stirring, and hypophosphorous acid (50% in aqueous solution, 0.17 g) is added. The vessel is heated up to 95-105° C. and vacuum is applied for 1 hour to remove the residual moisture. After bringing back to atmospheric pressure, under nitrogen flow, urea (0.6 g) is added and the vessel is heated up to 115-120° C. At this temperature, sulfamic acid (32.6 g) is added in about 2 hours. The reaction mixture is maintained at 115-120° C. until the percentage of SO₃ reaches a value of approximately 3%.

### Example 11

Ethoxylation (with 18 Moles of Ethylene Oxide) of the Diol of the Glycidyl Ester of Rosin Acids

In a stirred stainless steel reactor equipped for temperature measurement and control, pressure measurement, vacuum and inert gas purging, sampling and for introduction of ethylene oxide as a liquid, melted diol of the glicydyl ester of rosin acids (1108 g) and Potassium hydroxide 35% in aqueous solution (25.8 g) are added. The reactor content is heated to 120-125° C. and maintained under vacuum and nitrogen flow for 45 minutes to reduce its moisture content to less than 0.1%. The reactor is pressurized with nitrogen to 110-140 kPa and heated to 135° C. Ethylene oxide (2010 g) is then added while the temperature is maintained at 135-145° C. Following a 30 minutes period of digestion at reaction temperature, the reaction mixture is cooled to 80° C. and Acetic acid 80% in aqueous solution (12 g) is added.

### Example 12

### Phosphate Ester of the Product Prepared in Example 11

In a reaction vessel equipped with heating, stirrer, thermometer, a system of introduction of the reagents, such reaction vessel connected to a cooler provided of collector of water, the reaction product of Example 11 (671.23 g) is added. The reactor is heated to 60° C., under stirring, and hypophosphorous acid (50% in aqueous solution, 0.35 g) is added. The vessel is heated up to 95-105° C. and vacuum is applied for 1 hour to remove the residual moisture. The reaction mixture is then cooled down to 35-40° C. under nitrogen flow and phosphoric anhydride (25.36 g) is added in about 4 hours. Once phosphoric anhydride has been loaded completely the reaction mixture is maintained at 38-42° C. for about 30 minutes. Distilled water (4.46 g) is then added and the vessel is heated up to 55-60° C. The reaction mixture is maintained at 55-60° C. until the acid number reaches a value of approximately 45 mg KOH/g.

### Example 13

### Phosphate Ester (Triethanolamine Salt) of the Product Prepared in Example 12

In a reaction vessel equipped with heating, stirrer, thermometer, a system of introduction of the reagents, such reaction vessel connected to a cooler, the reaction product of Example 12 (320 g) is added. The reactor is heated to 40-50° C., under stirring, and triethanolamine (40 g) is added keeping under stirrer for 30 minutes.

### Example 14

### Sulfate Ester (Ammonium Salt) of the Product Prepared in Example 11

In a reaction vessel equipped with heating, stirrer, thermometer, a system of introduction of the reagents, such reaction vessel connected to a cooler provided of collector of water, the reaction product of Example 11 (325.95 g) is added. The reactor is heated to 60° C., under stirring, and hypophosphorous acid (50% in aqueous solution, 0.17 g) is added. The vessel is heated up to 95-105° C. and vacuum is applied for 1 hour to remove the residual moisture. After bringing back to atmospheric pressure, under nitrogen flow, urea (0.6 g) is added and the vessel is heated up to 115-120° C. At this temperature, sulfamic acid (24.04 g) is added in about 2 hours. The reaction mixture is maintained at 115-120° C. until the active content percentage of SO₃ reaches a value of approximately 3%.

### Example 15

### Phosphate Ester of the Product Prepared in Example 1

In a reaction vessel equipped with heating, stirrer, thermometer, a system of introduction of the reagents, such reaction vessel connected to a cooler provided of collector of water, the reaction product of Example 1 (331.6 g) is added. The reactor is heated to 60° C. under stirring and hypophosphorous acid 50% in aqueous solution (0.17 g) is added. The vessel is heated up to 95-105° C. and vacuum is applied for 1 hour to remove the residual moisture. The reaction mixture is then cooled down to 35-40° C. under nitrogen flow and phosphoric anhydride (16.7 g) is added in about 4 hours. Once phosphoric anhydride has been loaded completely, the reaction mixture is maintained at 38-42° C. for about 30 minutes. Distilled water (2.23 g) is then added and the vessel is heated up to 55-60° C. The reaction mixture is maintained at 55-60° C. until the acid number reaches a value of approximately 55 mg KOH/g.

### Example 16

### Phosphate Ester of the Product Prepared in Example 4

In a reaction vessel equipped with heating, stirrer, thermometer, a system of introduction of the reagents, such reaction vessel connected to a cooler provided of collector of water, the reaction product of example 4 (327.6 g) is added. The reactor is heated to 60° C. under stirring and hypophosphorous acid 50% in aqueous solution (0.17 g) is added. The vessel is heated up to 95-105° C. and vacuum is applied for 1 hour to remove the residual moisture. The reaction mixture is then cooled down to 35-40° C. under nitrogen flow and phosphoric anhydride (20.69 g) is added in about 4 hours. Once phosphoric anhydride has been loaded completely, the reaction mixture is maintained at 38-42° C. for about 30 minutes. Distilled water (1.53 g) is then added and the vessel is heated up to 55-60° C. The reaction mixture is maintained at 55-60° C. until the acid number reaches a value of approximately 65 mg KOH/g.

### Comparative Example A

Ethoxylated (with 20 moles of ethylene oxide) tristyrylphenol phosphate, salt with triethanolamine.

### Comparative Example B

Ethoxylated (with 20 moles of ethylene oxide) tristyrylphenol.

### Applicative Tests

### Suspension Concentrates

In order to evaluate the performance as dispersing agents of the surfactants of the present invention, suspension concentrates (SC) were prepared.

Suspension concentrates were prepared according to the following procedure. Monopropylene glycol (MPG) and dispersing agent are mixed together to form a homogeneous solution in a beaker. Water and defoamer are added and the solution is stirred till is limpid. The agrochemically active ingredient is added. The obtained suspension is mixed by using an high shear mixer (Ultra Turrax) for at least ten minutes. The suspension is milled with DYNO®-MILL KD till particle size of active ingredients is less than 5 microns, measured as D(90). The suspension concentrate is completed by adding a xanthan gum to get a Brookfield viscosity from 700 to 1800 mPa*s at 20 rpm, 20° C.

Different suspension concentrates were prepared using chlorotalonil (Table 1) or difenoconazole (Table 2) as agrochemically active ingredient. The amounts reported in Tables 1 and 2 are % by weight (wt %).

The defoamer used in the preparation of the suspension concentrates is SILFOAM® SE 47, commercially available from Wacker.

TABLE 1

| | wt % |
|---|---|
| Chlorothalonil | 40 |
| MPG | 5 |
| Dispersing agent | 5 |
| SILFOAM ® SE 47 | 0.5 |
| Distilled water | 39.5 |
| Xanthan gum | 10 |

17 suspension concentrates of chlorotalonil were prepared, using as dispersing agents the surfactant of Comparative Example A or surfactants of Examples 1-16. The obtained suspension concentrates are labelled as CSC-A, CSC-1, CSC-2, CSC-3, CSC-4, CSC-5, CSC-6, CSC-7, CSC-8, CSC-9, CSC-10, CSC-11, CSC-12, CSC-13, CSC-14, CSC-15 and CSC-16.

TABLE 2

| | wt % |
|---|---|
| Difenoconazole | 24.8 |
| MPG | 5 |
| Dispersing agent | 2.5 |
| SILFOAM ® SE 47 | 0.5 |
| Distilled water | 53.2 |
| Xanthan gum | 14 |

7 suspension concentrates of difenoconazole were prepared, using as dispersing agents the surfactant of Comparative Example A or surfactants of Examples 1, 3, 4, 5, 10 and 14. The obtained suspension concentrates are labelled as DSC-A, DSC-1, DSC-3, DSC-4, DSC-5, DSC-10 and DSC-14.

The following characteristics of the suspension concentrates were measured:
Particle size D(90)

Particle size D(90) in micron, corresponding to 90% of the cumulative distribution by volume as defined by standard method ISO 13320-1, with a Malvern Mastersizer Hydro 2000S. Evaluated just after the preparation of the suspension concentrate, after 1 week at 0° C., and after 2 weeks at 54° C.
Suspensibility (CIPAC Method MT 184)

Suspensibility of aqueous suspension concentrates; it involves preparing 250 ml of aqueous diluted suspension concentrate mixed with thirty inversions of the measuring cylinder, allowing it to stand for 1 hour in the cylinder under defined conditions, and removing the top nine-tenths. The remaining tenth is then assayed essayed either chemically, gravimetrically or by solvent extraction. The method gives an index of the stability of the homogeneity of the diluted suspension concentrate over time. Complete stability of the homogeneity corresponds to 100%.
Spontaneity of dispersion (CIPAC Method MT 187)

Spontaneity of dispersion of suspension concentrates; it is based on the preparation of 250 ml of a mixture of the suspension concentrate and water, mixed with only one inversion of the measuring cylinder. After standing 5 minutes under defined conditions the top nine-tenths is removed, and the remaining tenth assayed chemically, gravimetrically or by solvent extraction. The method gives an index of the immediate homogeneity of the diluted suspension concentrate. Complete immediate homogeneity corresponds to 100%.
Storage stability at 54° C. (CIPAC Method MT 46.3) and at 0° C. (CIPAC Method MT 39.3)

The storage stability of the suspension concentrates described above was evaluated at 0° C. (according to test method CIPAC 39.3), at room temperature and at 54° C. (according to test method CIPAC 46.3) in order to monitor the presence or absence of formation of an oil phase or of a creamy phase in the composition, the presence or absence of the occurrence of aggregation or precipitation, and the presence or absence of the formation of a supernatant over time. About 100 ml of the agrochemical suspension concentrates were sealed in glass containers and allowed to rest at 0° C. for 1 week and in oven at 54° C. for 2 weeks.

The results of the applicative tests on the suspensions concentrates are reported in Table 3 and in Table 4.

TABLE 3

| | Su | Sp | D90 | D90 (0° C.) | D90 (54° C.) | Stability at | Stability at |
|---|---|---|---|---|---|---|---|
| | (%) | (%) | (micron) | (micron) | (micron) | 0° C. | 54° C. |
| CSC-A* | 98.9 | 98.8 | 3.0 | 3.7 | 5.6 | stable | stable |
| CSC-1 | 99.7 | 99.8 | 2.0 | 1.5 | 2.9 | stable | stable |
| CSC-2 | 99.4 | 99.1 | 3.3 | 2.9 | 2.9 | stable | stable |
| CSC-3 | 99.8 | 99.6 | 2.8 | 2.1 | 2.4 | stable | stable |
| CSC-4 | 98.9 | 99.2 | 3.1 | 2.5 | 2.8 | stable | stable |
| CSC-5 | 99.7 | 99.0 | 2.5 | 2.5 | 2.5 | stable | stable |
| CSC-6 | 99.2 | 99.5 | 2.9 | 2.3 | 2.4 | stable | stable |
| CSC-7 | 99.4 | 98.9 | 1.9 | 1.9 | 2.2 | stable | stable |
| CSC-8 | 99.2 | 99.5 | 2.0 | 1.9 | 2.0 | stable | stable |
| CSC-9 | 99.4 | 99.4 | 2.7 | 2.5 | 2.2 | stable | stable |
| CSC-10 | 99.6 | 99.2 | 2.3 | 2.0 | 3.0 | stable | stable |
| CSC-11 | 99.4 | 99.1 | 2.6 | 2.4 | 2.8 | stable | stable |
| CSC-12 | 99.0 | 99.3 | 1.9 | 2.2 | 3.0 | stable | stable |
| CSC-13 | 99.6 | 99.4 | 2.6 | 1.4 | 2.6 | stable | stable |
| CSC-14 | 99.4 | 99.0 | 2.1 | 1.7 | 2.0 | stable | stable |
| CSC-15 | 99.2 | 99.1 | 1.9 | 1.4 | 2.1 | stable | stable |
| CSC-16 | 99.4 | 99.6 | 2.6 | 1.5 | 2.4 | stable | stable |

*Comparative
Su: Suspensibility
Sp: Spontaneity
D90: particle size measured just after the preparation of the suspension concentrate
D90 (0° C.): particle size measured after storage for 1 week at 0° C.
D90 (54° C.): particle size measured after storage for 2 weeks at 54° C.

TABLE 4

| | Su | Sp | D90 | D90 (0° C.) | D90 (54° C.) | Stability at | Stability at |
|---|---|---|---|---|---|---|---|
| | (%) | (%) | (micron) | (micron) | (micron) | 0° C. | 54° C. |
| DSC-A* | 99.8 | 99.5 | 2.8 | 2.4 | 3.2 | stable | stable |
| DSC-1 | 99.9 | 99.9 | 3.7 | 2.9 | 3.4 | stable | stable |
| DSC-2 | 99.9 | 99.6 | 3.4 | 3.7 | 2.3 | stable | stable |
| DSC-3 | 99.9 | 99.8 | 3.7 | 2.9 | 3.1 | stable | stable |
| DSC-4 | 99.9 | 99.9 | 4.9 | 5.6 | 3.8 | stable | stable |
| DSC-5 | 99.1 | 99.5 | 3.0 | 2.3 | 2.4 | stable | stable |
| DSC-6 | 99.2 | 98.9 | 2.6 | 2.8 | 3.0 | stable | stable |

*Comparative
Su: Suspensibility
Sp: Spontaneity
D90: particle size measured just after the preparation of the suspension concentrate
D90 (0° C.): particle size measured after storage for 1 week at 0° C.
D90 (54° C.): particle size measured after storage for 2 weeks at 54° C.

### Emulsifiable Concentrates

In order to evaluate the performance as emulsifiers of the surfactants of the present invention, emulsifiable concentrates (EC) were prepared.

Emulsifiable concentrates were prepared according to the following procedure.

The emulsifier was added to the solvent Solvesso 150 or Solvesso 120 (aromatic hydrocarbon oils, commercially available from ExxonMobil) while stirring. Then the agrochemically active ingredient was added under mechanical agitation until a clear solution was obtained.

Different emulsifiable concentrates were prepared using S-metholachlor (Table 5) or quizalofop-p-ethyl (Table 6) as agrochemically active ingredient. The amounts reported in Tables 5 and 6 are % by weight (wt %).

TABLE 5

| | wt % |
|---|---|
| S-metholachlor | 90.1 |
| Solvesso 200 | 2.6 |
| Emulsifier | 5.5 |
| Emulson ® AG/CAL 70 | 1.8 |

Emulson ® AG/CAL 70 is calcium dodecylbenzene sulfonate (active matter 70%), commercially available from Lamberti S.p.a.

5 emulsifiable concentrates of S-metholachlor were prepared, using as emulsifiers the surfactant of Comparative Example B or surfactants of Examples 1, 4, 7 and 11. The obtained emulsifiable concentrates are labelled as MEC-B, MEC-1, MEC-4, MEC-7 and MEC-11.

TABLE 6

| | wt % |
|---|---|
| Quizalofop-p-ethyl | 13.0 |
| Solvesso 150 | 79.0 |
| Emulsifier | 4.0 |
| Emulson ® AG CAL/E | 4.0 |

Emulson ® AG CAL/E is calcium dodecylbenzene sulfonate (active matter 60%), commercially available from Lamberti S.p.a.

3 emulsifiable concentrates of Quizalofop-p-ethyl were prepared, using as emulsifiers the surfactant of Comparative Example B or surfactants of Examples 1 and 4. The obtained emulsifiable concentrates are labelled as QEC-B, QEC-1 and QEC-4.

The following characteristics of the emulsifiable concentrates were measured:
Emulsion stability (CIPAC Method MT 36.1)

The emulsifiable concentrate (EC) were diluted with hard water (342 ppm) at 1 wt % dilution.

The emulsions are considered stable when no phase separation and less than 3% of cream at the top of the emulsion are observed.
Storage stability at 54° C. (CIPAC Method MT 46.4) and at 0° C. (CIPAC Method MT 39.3) The storage stability of the emulsifiable concentrates described above was evaluated at 0° C. (according to test method CIPAC 39.3), at room temperature and at 54° C. (according to test method CIPAC 46.3) in order to monitor the presence or absence of formation of an oil phase or of a creamy phase in the composition, the presence or absence of the occurrence of aggregation or precipitation, and the presence or absence of the formation of a supernatant over time. About 100 ml of the agrochemical emulsifiable concentrates were sealed in glass containers and allowed to rest at 0° C. for 1 week, and in oven at 54° C. for 2 weeks.

The results of the applicative tests on the emulsifiable concentrates are reported in Table 7 and in Table 8.

TABLE 7

| | Emulsion | Stability at | Stability at |
|---|---|---|---|
| | stability | 0° C. | 54° C. |
| MEC-B* | yes | stable | stable |
| MEC-1 | yes | stable | stable |
| MEC-4 | yes | stable | stable |
| MEC-7 | yes | stable | stable |
| MEC-11 | yes | stable | stable |

*Comparative
yes = a stable emulsion is formed

TABLE 8

| | Emulsion | Stability at | Stability at |
|---|---|---|---|
| | stability | 0° C. | 54° C. |
| QEC-B* | yes | stable | stable |
| QEC-1 | yes | stable | stable |
| QEC-4 | yes | stable | stable |

*Comparative
yes = a stable emulsion is formed

The results of the applicative tests show that the surfactants of the invention have excellent properties as dispersing agents and emulsifiers. Their performances are comparable with those of know petroleum-derived tristyrylphenol-based surfactants.

1.-10. (canceled)

11. A surfactant of Formula (I): 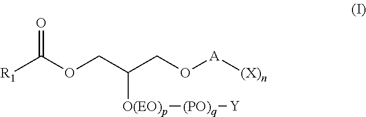
wherein:
R₁=residue derived from a rosin acid
$$X = H , {C}_{1} - {C}_{20} - alkyl , P = O ( {OR}_{2} ) ( {OR}_{3} ) or {SO}_{3} {R}_{4}$$
$$Y = H , P = O ( {OR}_{2} ) ( {OR}_{3} ) , {SO}_{3} {R}_{4}$$
$$A = ( EO ) r - ( PO ) s$$
R₂, R₃ and R₄=hydrogen, an alkali metal, ammonium ion, a protonated amine, a quaternary organic ammonium or an alkaline earth metal
$$EO = {CH}_{2} {CH}_{2} O$$
$$PO = {CH}_{2} CH ( {CH}_{3} ) O$$
n is an integer number ranging from 1 to 10
p and q are integer numbers ranging from 0 to 100
r and s are integer numbers ranging from 0 to 100, with the proviso that the sum r+s is at least 1.

12. The surfactant of Formula (I) according to claim 1, wherein p and q are integer numbers ranging from 2 to 40.

13. The surfactant of Formula (I) according to claim 1, wherein p and q are integer numbers ranging from 4 to 20.

14. The surfactant of Formula (I) according to claim 1, wherein r and s are integer numbers ranging from 2 to 40.

15. The surfactant of Formula (I) according to claim 1, wherein r and s are integer numbers ranging from 4 to 20.

16. The surfactant of Formula (I) according to claim 1, wherein R₁ is a residue derived from a rosin acid selected among abietic acid, palustric acid, dehydroabietic acid, neo-abietic acid, levo-pimaric acid, pimaric acid, sandaracopimaric acid, isopimaric acid or mixtures thereof.

17. An agrochemical composition comprising:
a. at least one agrochemically active ingredient;
b. at least one surfactant of Formula (I): 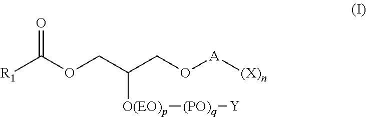
wherein:
R₁=residue derived from a rosin acid
$$X = H , {C}_{1} - {C}_{20} - alkyl , P = O ( {OR}_{2} ) ( {OR}_{3} ) or {SO}_{3} {R}_{4}$$
$$Y = H , P = O ( {OR}_{2} ) ( {OR}_{3} ) , {SO}_{3} {R}_{4}$$
$$A = ( EO ) r - ( PO ) s$$
R₂, R₃ and R₄=hydrogen, an alkali metal, ammonium ion, a protonated amine, a quaternary organic ammonium or an alkaline earth metal
$$EO = {CH}_{2} {CH}_{2} O$$
$$PO = {CH}_{2} CH ( {CH}_{3} ) O$$
n is an integer number ranging from 1 to 10.

18. The agrochemical composition according to claim 7, wherein p and q are integer numbers ranging from 2 to 40.

19. The agrochemical composition according to claim 7, wherein p and q are integer numbers ranging from 4 to 20.

20. The agrochemical composition according to claim 7, wherein r and s are integer numbers ranging from 2 to 40.

21. The agrochemical composition according to claim 7, wherein r and s are integer numbers ranging from 4 to 20.

22. The agrochemical composition according to claim 7, wherein R₁ is a residue derived from a rosin acid selected among abietic acid, palustric acid, dehydroabietic acid, neo-abietic acid, levo-pimaric acid, pimaric acid, sandaracopimaric acid, isopimaric acid or mixtures thereof.

23. The agrochemical composition according to claim 7, wherein the agrochemical composition comprises:
a. from 0.1 to 99 wt % (% by weight) of at least one agrochemically active ingredient;
b. from 0.01 to 40 wt % (% by weight) of at least one surfactant of Formula (I).

24. The agrochemical composition according to claim 7, wherein the agrochemically active ingredient is selected among fungicides, bactericides, insecticides, acaricides, nematicides, herbicides, aracnicides, insect growth regulators, repellents, plant growth regulators, plant nutrients or mixtures thereof.
